(19) United States
(12) Patent Application Publication
(10) Pub. No.: US 2010/0151575 A1
(43) Pub. Date: Jun. 17, 2010

(54) Method of Making Conditioned Media from Kidney Derived Cells

(76) Inventors: David C. Colter, Hamilton, NJ (US); Christian Kazanecki, Martins Creek, PA (US); Brian C. Kramer, Plainfield, NJ (US); Jackie J. Donners, West Windsor, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/335,186

(22) Filed: Dec. 15, 2008

(51) Int. Cl. C12N 5/02

(52) U.S. Cl. 435/395; 435/398

(57) ABSTRACT

Methods of making conditioned media by culturing cells on nonwoven substrates are disclosed. More specifically, methods of making conditioned media by culturing mammalian kidney-derived cells on nonwoven substrates are disclosed.

### FIELD OF THE INVENTION

The invention relates to methods of making conditioned media. Specifically, this invention relates to methods of making conditioned media by culturing cells on different substrates.

### BACKGROUND OF THE INVENTION

Evidence suggests that after the administration of multipotent stem and progenitor cells, the cells home to the sites of injury where they then differentiate and incorporate into the injured tissue. However, experimental evidence is increasingly supporting an alternate mechanism in which cytokines, secreted by the administered cells, stimulate tissue repair and regeneration. Modulation of growth factor and cytokine release would be a useful attribute for creating designer cellular therapeutics to treat specific diseases.

The above discrepancy is exemplified by current research on mesenchymal stem cells (MSCs). The therapeutic capacity of MSCs to treat a wide spectrum of diseases has been attributed to their potential to differentiate into many different reparative cell types. However, the efficiency of transplanted MSCs to differentiate into functional reparative cells within injured tissues or organs has not been adequately documented or demonstrated. Recent reports have suggested that some of these reparative effects are not mediated by differentiation of MSCs, but rather by paracrine factors secreted by MSCs (Caplan, A I & Dennis, J E: Mesenchymal stem cells as trophic mediators. J Cell Biochem, 98: 1076-1084, 2006.). These factors are postulated to promote angiogenesis; support the stem cell crypt in the intestine; protect against renal (Togel, F, Weiss, K, Yang, Y, Hu, Z, Zhang, P & Westenfelder, C: Vasculotropic, paracrine actions of infused mesenchymal stem cells are important to the recovery from acute kidney injury. Am J Physiol, 292: F1626-1635, 2007 and Bi, B, Schmitt, R, Israilova, M, Nishio, H & Cantley, L G: Stromal cells protect against acute tubular injury via an endocrine effect. J Am Soc Nephrol, 18: 2486-2496, 2007.), myocardial, and limb tissue injury. In addition, the secretion proteome of embryonic stem cell-derived MSC conditioned medium has recently been defined (Sze, S K, de Kleijn, D P V, Lai, R C, Tan, E K W, Zhao, H, Yeo, K S, Low, T Y, Lian, Q, Lee, C N, Mitchell, W, El Oakley, R M & Lim, S K: Elucidating the secretion proteome of human embryonic stem cell-derived mesenchymal stem cells. Mol & Cell Proteomics, 6: 1680-1689, 2008.)

The use of secreted factors in the form of conditioned medium will introduce a radically different dimension to the use of cell-based therapies in regenerative medicine. Instead of using cells, repair of injured tissues will be mediated by enhancing endogenous tissue repair mechanisms using biologics secreted by the various types of stem cells. This will bypass the present confounding issues associated with cell-based therapy, i.e. immune compatibility, tumorgenicity, infections, costs, and waiting time for ex vivo expansion of autologous cell preparations. Such an approach will have a greater potential for the development of “off-the-shelf” cell-based therapeutics, at affordable costs and with better quality control and consistency.

The commonly used methodology of generating conditioned medium is to use standard tissue culture techniques and plastic growth surfaces for the culture of cells. However, it is known that implanted biomaterials are able to alter secreted cytokine production by cells. Such reports have primarily examined immune cell responses, such as activation of dhendritic cells and macrophages (Jones, J A, Chang, D T, Meyerson, H, Colton, E, Kwon, I K, Matsuda, T & Anderson, J M: Proteomic analysis and quantification of cytokines and chemokines from biomaterial surface-adherent macrophages and foreign body giant cells. J Biomed Mat Res A, 83: 585-596, 2007.) or gene expression by multinucleated giant cells during the foreign body reaction to the implanted materials (Jones, J A, Chang, D T, Meyerson, H, Colton, E, Kwon, I K, Matsuda, T & Anderson, J M: Proteomic analysis and quantification of cytokines and chemokines from biomaterial surface-adherent macrophages and foreign body giant cells. J Biomed Mat Res A, 83: 585-596, 2007 and Luttikhuizen, D T, Dankers, P Y, Harmsen, M C & van Luyn, M J: Material dependent differences in inflammatory gene expression by giant cells during the foreign body reaction. J Biomed Mat Res A, 83: 879-886, 2007.). These research efforts have used cytokine production as a way to screen for biomaterials that would decrease the production of inflammatory molecules leading to increased biocompatibility.

In this application, we describe the novel use of non-woven biomaterial substrates, of different composition and density, for the purpose of altering the production of desirable cytokines in cell conditioned medium samples used for therapy. Our results support the idea that the choice of biomaterial substrate used for cell culture, when generating conditioned medium, will ultimately lead to an increase in cytokine concentration and efficacy of the conditioned medium.

It is well known that exogenously added growth factors, cytokines and chemokines influence cell behavior and the regulation of soluble factor secretion. However, the effects of a synthetic cell attachment substrate on the secretory behavior of cells in not well understood. The experiments that we describe here demonstrate that the polymer chemistry and density of a cell attachment substrate influences the amount of various growth factors/cytokines secreted from the cell. The polymer chemistry of the scaffolds will affect the type and amount of proteins that are able to absorb onto the scaffold surface, in turn affecting cell signaling mechanisms and subsequent secreted factor production. The density of the scaffolds greatly affects the 3D architecture that the cell is able to perceive, which will also affect secreted factor production through non-specific signaling mechanisms. In summary, the observed effects may be mediated by non-specific cell signaling mechanisms, as well as through cell surface receptors and intracellular signaling pathways. These findings demonstrate that growing cells on a synthetic polymer scaffolds will allow for novel and therapeutically relevant conditioned media to be produced, and are the first step towards the design of engineered surfaces for production of conditioned medium with maximum efficiency.

### SUMMARY OF THE INVENTION

We have provided a method of making conditioned media by culturing cells on nonwoven substrates. Culturing cells on nonwoven substrates alters the cytokine expression profile of the cells as compared to the cytokine expression profile on tissue culture plastic. As a result, we have provided a method of producing conditioned media that is enriched in selective cytokines when grown on nonwoven substrates.

### BRIEF DESCRIPTION OF THE DRAWINGS

FIG. 1: Average number of cells recovered from non-woven substrate samples. One centimeter diameter samples were seeded with 20,000 hKDCs and cultured for seven days. Conditioned medium was generated by overnight incubation, cells were recovered using trypsinization and counted using the Guava instrument. Data represent the mean number of cells from quadruplicate samples. Error bars represent standard deviation

FIG. 2: Average number of cells recovered from non-woven substrates. One centimeter diameter samples were seeded with 20,000 hKDCs and cultured for seven days. Conditioned medium was generated by overnight incubation, and cell number determined using the CyQuant NF assay (Invitrogen). Data represent the mean number of cells from triplicate samples. Error bars represent standard deviation.

FIG. 3: Combined normalized ELISA results for a single substrate expressed as fold change compared to tissue culture plastic. The graph represents the range of fold change observed in the two examples for a 90/10 PGA/PLA (300 mg/mL) nonwoven substrate.

FIG. 4: Combined normalized ELISA results for a single substrate expressed as fold change compared to tissue culture plastic. The graph represents the range of fold change observed in the two examples for a 90/10 PGA/PLA (150 mg/mL) nonwoven substrate.

FIG. 5: Combined normalized ELISA results for a single substrate expressed as fold change compared to tissue culture plastic. The graph represents the range of fold change observed in the two examples for a 90/10 PGA/PLA (50 mg/mL) nonwoven substrate.

FIG. 6: Combined normalized ELISA results for a single substrate expressed as fold change compared to tissue culture plastic. The graph represents the range of fold change observed in the two examples for a 95/5 PLA/PGA (155 mg/mL) nonwoven substrate.

FIG. 7: Combined normalized ELISA results for a single substrate expressed as fold change compared to tissue culture plastic. The graph represents the range of fold change observed in the two examples for a 95/5 PLA/PGA (67 mg/mL) nonwoven substrate.

FIG. 8: Combined normalized ELISA results for a single substrate expressed as fold change compared to tissue culture plastic. The graph represents the range of fold change observed in the two examples for a 50% (90/10 PGA/PLA)/50% PDO (250/323 mg/mL) nonwoven substrate.

FIG. 9: Combined normalized ELISA results for a single substrate expressed as fold change compared to tissue culture plastic. The graph represents the range of fold change observed in the two examples for a 50% (90/10 PGA/PLA)/50% PDO (100/150 mg/mL) nonwoven substrate.

### DETAILED DESCRIPTION OF THE INVENTION

Conditioned medium is a medium in which a specific cell or population of cells has been cultured, and then removed. Conditioned medium may also be referred to herein as “conditioned media”. As used herein the term “population of cells” means one or more cells. While the cells are cultured in the medium, they secrete cellular factors that can provide trophic support to other cells. Such trophic factors include, but are not limited to hormones, cytokines, extracellular matrix (ECM), proteins, antibodies, and granules. The medium containing the cellular factors is the conditioned medium. We have disclosed a method of making conditioned media by providing a population of mammalian kidney-derived cells (KDCs), then seeding the cells on a nonwoven substrate, the cells are subsequently grown in culture on the nonwoven substrate in renal growth medium, the renal growth medium is removed, serum free medium is added to the cells and the cells are grown in culture for about 24 hours. The conditioned medium is then isolated from the cell culture.

We have demonstrated dramatic increases in the amount of secreted soluble factors when KDC cells are cultured on non-woven polyester scaffolds compared to culture on standard tissue culture plastic. We have also shown that conditioned medium from KDC cells is able decrease apoptosis in an in vitro assay.

The mammalian kidney-derived cells (KDCs) are provided as described in US patent publication number 20080112939, incorporated by reference herein in its entirety. An isolated or purified mammalian kidney-derived cell population is provided, said cell population capable of self-renewal and expansion in culture, wherein the cell population is positive for expression of at least one of Oct-4, Rex-1, Pax-2, Cadherin-11, FoxD1, WT1, Eyal, HNF3B, CXC-R4, Sox-17, EpoR, BMP2, BMP7, or GDF5 and negative for the expression of at least one of Sox2, FGF4, hTert, Wnt-4, SIX2 or GATA-4. The isolated or purified mammalian kidney-derived cell population is stable and capable of self-renewal and expansion in cell culture. In one embodiment, the cell is positive for expression of at least one of Eyal, WT1, FoxD1, BMP7, BMP2, GDF5, EpoR or Rex-1, and negative for expression of at least one of Sox2, FGF4, hTert or Wnt-4. The cell population is non-immunogenic for allogeneic transplantation in a mammalian subject, as evidenced by the finding that the isolated or purified mammalian kidney-derived cell population is positive for the cell-surface marker HLA I, and negative for at least one of cell-surface markers HLA II, CD80, or CD86. The mammalian kidney-derived cell population may secrete at least one of trophic factors FGF2, HGF, TGFβ, TIMP-1, TIMP-2, MMP-2 or VEGF and does not secrete at least one of trophic factors PDGF-bb or IL12p70. The cell population can be derived from kidney subcapsular region, kidney cortex, or from kidney medulla, from a human, a primate, or a rodent. The mammalian kidney-derived cell population may include kidney progenitor cells.

The mammalian kidney-derived cells provided as described above are seeded on a nonwoven substrate. The mammalian kidney-derived cells may be obtained from about passage 3 to about passage 10. In one embodiment, the mammalian kidney-derived cells may be obtained from about passage 7. The cells are suspended in growth medium (GM) in the amount of about 10,000 cells/mL to about 1 million cells/mL of GM. In one embodiment, the cells are suspended in the amount of about 20,000 cells/mL of GM. The suspended cells are then seeded on a nonwoven substrate and grown for about 2 to about 7 days in culture with fresh GM exchange every 2-3 days. In one embodiment, the cells are grown on the nonwoven substrate for about 7 days.

The nonwoven substrate is a nonwoven fabric or felt. The term “nonwoven fabric” includes, but is not limited to, bonded fabrics, formed fabrics, or engineered fabrics, that are manufactured by processes other than, weaving or knitting. More specifically, the term “nonwoven fabric” refers to a porous, textile-like material, usually in flat sheet form, composed primarily or entirely of fibers, such as staple fibers assembled in a web, sheet or batt. The structure of the nonwoven fabric is based on the arrangement of, for example, staple fibers that are typically arranged more or less randomly.

Nonwoven fabrics can be created by a variety of techniques known in the textile industry. The methods create carded, wet laid, melt blown, spunbonded, or air laid, nonwovens. In the carded non-wovens process, short fibers, known as staple fibers, are combed into a web by passing through rotating cylinders covered by wires with teeth. The fibers used to make the nonwoven fabric can be monofilaments, yarns, threads, braids, or bundles of fibers. The fibers may be kinked or piled. Carded nonwovens tend to have a predominantly uni-directional fiber orientation. The wet laid non-wovens process begins with a slurry, typically consisting of a high percentage of water and staple fibers. The slurry is collected on a screen, which can be a wire belt on an incline, a cylinder, or fed between two wire belts. The water is removed by squeezing the web between rolls, and dried in ovens. Wet laid nonwovens are isotropic (equal machine and cross-directional strength), strong, highly uniform and can be quite absorbent with excellent wicking properties. The non-woven fabrics may be strengthened by a number of techniques including thermal bonding, chemical bonding, or hydroentangling, and needlepunching. Thermally bonding a nonwoven involves applying heat and pressure via a calendar to consolidate the web. Chemical or resin bonding involves applying an adhesive resin (binder) to the fabric by dipping it in a bath, or spraying, foaming or printing the binder onto the web. The binder solution is removed by drying the web. Hydroentangling uses water through fine, high pressure jets which cause the fibers to curl and entangle about each other. Needlepunched non-wovens are consolidated by inserting barbed needles mechanically into the non-woven fabric, hooking tufts of fibers through it and entangling the fibers in the needlepunched areas. During this process, the fabric travels between two plates while it is pulled by draw rolls. Needlepunched nonwovens are typically strong and heavier than most other nonwovens products.

The density of the nonwoven fabrics may be varied depending upon the processing conditions. In one embodiment, the nonwoven fabrics have a density of about about 60 mg/mL to about 350 mg/mL.

The fibers used to make the nonwoven fabric may be biomaterials comprised of biocompatible, bioabsorbable polymers. Examples of suitable biocompatible, bioabsorbable polymers that could be used include polymers selected from the group consisting of aliphatic polyesters, poly(amino acids), copoly(ether-esters), polyalkylene oxalates, polyamides, poly(iminocarbonates), polyorthoesters, polyoxaesters, polyamidoesters, polyoxaesters containing amine groups, poly(anhydrides), polyphosphazenes, and blends thereof.

In one embodiment, the aliphatic polyesters are homopolymers and/or copolymers of monomers selected from the group consisting of lactide (which includes lactic acid, D-,L- and meso lactide), glycolide (including glycolic acid), epsilon-caprolactone, p-dioxanone (1,4-dioxan-2-one), trimethylene carbonate (1,3-dioxan-2-one), alkyl derivatives of trimethylene carbonate, delta-valerolactone, beta-butyrolactone, gamma-butyrolactone, epsilon-decalactone, hydroxybutyrate (repeating units), hydroxyvalerate (repeating units), 1,4-dioxepan-2-one (including its dimer 1,5,8,12-tetraoxacyclotetradecane-7,14-dione), 1,5-dioxepan-2-one, 6,6-dimethyl-1,4-dioxan-2-one and polymer blends thereof. In another embodiment, aliphatic polyesters which include, but are not limited to homopolymers and/or copolymers of lactide (which includes lactic acid, D-,L- and meso lactide), glycolide (including glycolic acid), epsilon-caprolactone, p-dioxanone (1,4-dioxan-2-one), trimethylene carbonate (1,3-dioxan-2-one) and combinations thereof.

In another embodiment, the aliphatic polyesters are homopolymers and/or copolymers of monomers selected from the group consisting of lactide (which includes lactic acid, D-,L- and meso lactide), glycolide (including glycolic acid), epsilon-caprolactone, p-dioxanone (1,4-dioxan-2-one), trimethylene carbonate (1,3-dioxan-2-one) and combinations thereof. In yet another embodiment, the aliphatic polyesters are homopolymers and/or copolymers of monomers selected from the group consisting of lactide (which includes lactic acid, D-,L- and meso lactide), glycolide (including glycolic acid), and p-dioxanone (1,4-dioxan-2-one) and combinations thereof.

Growth medium, generally refers to any substance or preparation used for the cultivation of living cells. In one embodiment the growth medium is renal growth medium. In another embodiment the growth medium is Dulbecco's Modification of Eagle's medium (DMEM).

Following the cell growth in the GM, the spent GM is removed from the cell culture and replaced with serum free growth medium. The cells are grown in the serum free growth medium for no more than about 24 hours. Serum free growth medium is Dulbecco's Modification of Eagle's medium (DMEM), and optionally may be phenol-red free DMEM. The conditioned serum free growth medium is subsequently isolated from the culture.

The conditioned free medium prepared by the methods described herein may be useful in the treatment of medical conditions. Our data strongly support the suggestion that culturing cells on non-woven biomaterials alters the composition of the resulting conditioned medium in a positive manner. This observation is extremely useful for the generation of conditioned medium with increased effectiveness for use as a therapeutic product. For example, the cytokine GM-CSF has been shown to be beneficial for spinal cord injury and after hepatectomy, and administration of the recombinant protein is currently used after chemotherapy to accelerate the recovery of white blood cells. Recombinant GM-CSF is sold under the tradename LEUKINE (Bayer Healthcare Pharmaceuticals, Montville, N.J.). Our results show increases of 6.4 to 35.9-fold GM-CSF production compared to TCP, depending on the biomaterial composition chosen. Hepatocyte growth factor (HGF) and vascular endothelial growth factor (VEGF) are factors involved in angiogenesis, wound healing and regeneration, particularly in the liver and kidney, and are being evaluated as a gene therapy targets (Nishino, M, Iimuro, Y, Ueki, T, Hirano, T, Fujitmoto, J: Hepatocyte growth factor improves survival after partial hepatectomy in cirrhotic rats suppressing apoptosis of hepatocytes. Surgery 144(3): 374-84, 2008 and Bao, P, Kodra, A, Tomic-Canic, M, Golinko, M S, Ehrlich, H P, Brem, H: The role of vascular endothelial growth factor in wound healing. Journal of Surgical Research, published online ahead of print doi: 10.1016/j.jss.2008.04.023, 2008). Again, our results show an average increase in HGF production of 1.2 to 4.1-fold and in VEGF production of 1.9 to 9.7-fold compared to TCP, demonstrating the advantages of culturing cells on non-woven biomaterials for conditioned medium production.

A distinct advantage of using conditioned medium isolated from cells is that the combination of factors secreted by the cells have the potential to have dramatically increased effects compared to using single molecules alone. The therapeutic effects of paracrine factors secreted by cells have been documented—especially for mesenchymal stem cells [1-4] and more recently for adipose-derived stem cells (Nakagami, H, Maeda, K, Morishita, R, Iguchi, S, Nishikawa, T, Takami, Y, Kikuchi, Y, Saito, Y, Tamai, K, Ogihara, T & Kaneda, Y: Novel autologous cell therapy in ischemic limb disease through growth factor secretion by cultured adipose tissue-derived stromal cells. Arterioscler Thromb Vasc Biol, 25: 2542-2547, 2005 and Wei, X, Du, Z, Zhao, L, Feng, D, Wei, G, He, Y, Tan, J, Lee, W-H, Hampel, H, Dodel, R, Johnstone, B H, March, K, L., Farlow, M R & Du, Y: IFATS Series: The Conditioned Media of Adipose Stromal Cells Protect Against Hypoxia-Ischemia-Induced Brain Damage in Neonatal Rats. Stem Cells, epub ahead of print, 2008). Many of the beneficial factors secreted by cells act in an additive fashion to protect cells from injury and promote repair and regeneration.

The following examples are illustrative of the principles and practice of this invention, although not limited thereto. Numerous additional embodiments within the scope and spirit of the invention will become apparent to those skilled in the art once having the benefit of this disclosure.

### EXAMPLE 1

Method of Making Cell culture Media on Nonwoven Substrates

This example illustrates that culturing cells on non-woven substrates increases the production of trophic factors, altering the composition of the resulting conditioned medium in a positive manner. The observations are a result of both the substrate composition as well as the fabric density.

### Cell Seeding and Culture

Circular substrates of one centimeter in diameter were made from nonwoven fabrics of various compositions. The nonwoven fabrics chosen are shown in Table 1. A nonwoven fabric comprising fibers of 90/10 poly(glycolide-co-lactide) (PGA/PLA) sold under the tradename VICRYL (Ethicon, Inc., Somerville, N.J.), a nonwoven fabric comprising fibers of 95/5 poly(lactide-co-glycolide) (PLA/PGA) sold under the tradename 95/5 PLA/PGA, and a nonwoven fabric comprising 50% (90/10 PGA/PLA) fibers and 50% PDO fibers were tested having different densities. Nonwoven fabrics comprising 90/10 PGA/PLA fibers and nonwoven fabrics comprising 50% (90/10 PGA/PLA) fibers and 50% PDO fibers were prepared by Concordia, (Coventry, R.I.), while nonwoven fabrics comprising fibers of 95/5 PLA/PGA were prepared by Albany International Techniweave, Inc. (Rochester, N.Y.) The substrates were placed in low-cluster 24-well plates and sterilized by soaking in 100% ethanol for four hours. The substrates were then washed with phosphate-buffered saline (PBS—Invitrogen) and placed in renal epithelial growth medium (REGM—Lonza, Walkersville).

Cells (human kidney-derived cells (hKDC) lot 032906p7) were thawed, counted and brought to 20,000 cells/ml in REGM. The medium was aspirated from the substrates and one milliliter of the cell suspension (20,000 cells) was added to the wells containing the substrate. In addition, 24-well tissue culture plates were seeded as a control (tissue culture plastic). The cell seeded substrates and control wells were cultured for seven days with fresh medium exchange every two to three days.

TABLE 1
Non-woven substrates assayed. One-centimeter circular samples were
created from each of the listed substrates.

| | Density | Thickness | |
|---|---|---|---|
| Substrate Composition | (mg/ml) | (mm) | Lot # |
| 100% 95/5 PLA/PGA | 67 | 1 | 5213-34-1 |
| 100% 95/5 PLA/PGA | 155 | 1 | 5213-34-4 |
| 100% 90/10 PGA/PLA | 60 | 1 | MD00287 rev2 |
| 100% 90/10 PGA/PLA | 150 | 1.5 | MD00092 rev1 |
| 100% 90/10 PGA/PLA | 300 | 1.5 | MD00082 rev3 |
| 50% (90/10 PGA/PLA)/ | 100 | 1 | MD00323-01 |
| 50% PDS | | | |
| 50% (90/10 PGA/PLA)/ | 250 | 1 | MD00021 rev2 |
| 50% PDS | | | |

### ELISA Analysis of Trophic Factor Secretion

On day seven post seeding, the cell-seeded substrates were washed twice with PBS (1 ml/wash), and 500 μl of serum free, phenol-free Dulbecco's Modified Eagles Medium (DMEM) (Invitrogen, Carlsbad, Calif.) supplemented with 2 nM Glutamax (Invitrogen) was added. The cell-seeded substrates were then cultured overnight to generate conditioned medium. The next day, the conditioned medium was collected into 1.5 ml low-retention tubes, centrifuged to remove cells and cell debris, and frozen at −80° C. The concentration of ten specific protein analytes (hTGFβ1, hGCSF, hGMCSF, hIL-4, hVEGF, hHGF, hM-CSF, hFGFβ, hMMP-7, hIL-6) was determined by ELISA (Pierce Biotechnology, Rockford, Ill.).

After removing the conditioned medium, the cell-seeded substrates were washed with PBS and cells were recovered with 0.5 ml Trypsin-EDTA (Invitrogen). Cells were removed from the substrates by adding 0.5 ml REGM, triturating to dislodge cells, and transferring to 15 ml conical tubes. The substrates were then rinsed with an additional REGM wash and the wash was then combined with the harvested cells. The cells were pelleted, resuspended in 0.5 ml REGM and counted using the Guava ViaCount assay and instrument (Guava Technologies, Hayward, Calif.). The resulting cell counts were then used to normalize the analyte protein concentrations.

### Cell Attachment and Growth

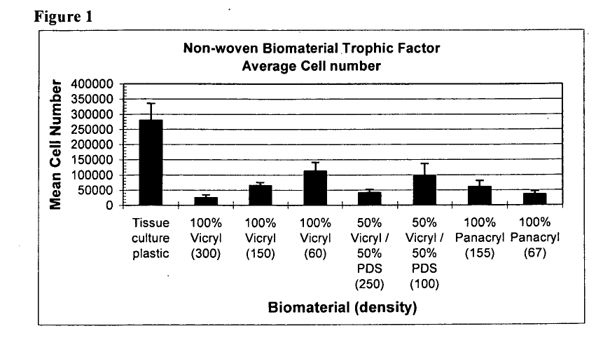

hKDCs were seeded onto one centimeter diameter samples of various non-woven substrates differing in composition and density and cultured for seven days. Conditioned medium was then harvested and sent for ELISA analysis. Cells growing on the substrates were recovered by trypsinization. FIG. 1 shows the average number of cells. Two of the three different compositions tested (100% 90/10 PGA/PLA and 50% 90/10 PGA/PLA/50% PDS) displayed the same trend—as the density of the substrate decreased, the number of cells recovered from the substrate increased. For example, the least dense 90/10 PGA/PLA (60 mg/ml) samples averaged almost 6 times more cells than the most dense sample (90/10 PGA/PLA 300 mg/ml). The 95/5 PLA/PGA samples showed the opposite result. More cells were recovered from the higher density sample than from the lower density sample. The tissue culture plastic control supported significantly more cell growth than all substrates tested an average of more than double the cells recovered than the highest substrate (100% 90/10 PGA/PLA 60 mg/ml).

### Analysis of Trophic Factor Secretion

Conditioned medium samples were analyzed by ELISA and the concentration of 10 different analytes were determined. The resulting analyte protein concentrations were normalized to the cell number recovered from the specific samples. Table 2 shows the normalized protein concentration for all analytes and substrates. In general, the expression of most analytes was greatly increased when cells are grown on substrates compared to tissue culture plastic (TCP). Only hMMP-7 was expressed at a higher concentration by cells growing on tissue culture plastic than the substrate samples. Composition of the non-woven substrates affected analyte production. For some analytes (i.e. hTGFβ-1) the 100% 90/10 PGA/PLA sample showed higher analytes production than the similar density 100% 95/5 PLA/PGA sample, yet for other analytes the opposite was observed (i.e. hHGF, hFGFβ). Some analytes also showed no difference between the two (hM-CSF).

Also, an important trend was observed indicating that the density of the non-woven substrate sample affects the analyte production per cell. The normalized data show that for most analytes examined, increasing the density of the substrate sample resulted in an increase in analyte production per cell. This is exemplified by the hVEGF and hHGF results, in which the analyte concentration per cell for the higher density samples of all three different compositions tested were significantly higher than that of the lowest density samples, which were more comparable to TCP. Table 3 shows the fold change in the normalized analyte expression compared to TCP for each substrate. With the single exception of MMP-7, the expression of all analytes were increased compared to TCP.

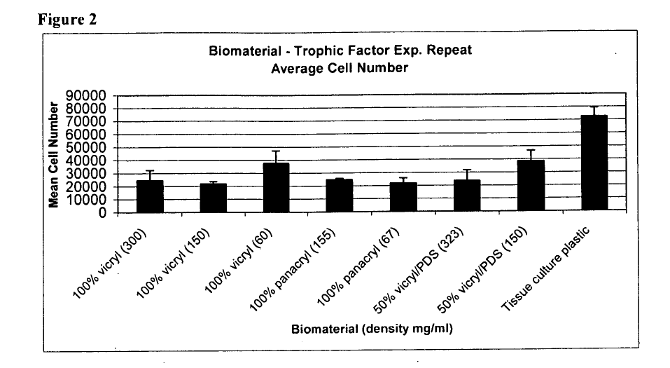

TABLE 2
Searchlight analysis of conditioned medium. Conditioned medium,
generated from hKDCs cultured on various non-woven substrates, were analyzed
using Searchlight protein array analysis. Data shown are the mean analyte
concentrations of duplicate samples normalized to cell number (pg/ml/10⁶ cells).
Data is shown in graphical form in FIG. 2. Samples labeled ‘UND’ indicate that
the analyte level was below the detectable limit of the ELISA assay.

| Non-Woven | hTGFB1 | hGCSF | hGMCSF | hIL4 | hVEGF | hHGF | hM-CSF | hFGFb | hMMP7 | hIL6 |
|---|---|---|---|---|---|---|---|---|---|---|
| Substrate & (density) | pg/ml/10e6 cells | | | | | | | | | |
| 100% 90/10 PGA/PLA (300) | 20472 | 353 | 19791 | 232 | 143375 | 7866 | 24233 | 1311 | 24074 | 119289 |
| 100% 90/10 PGA/PLA (150) | 18713 | 92 | 10652 | 37 | 65986 | 4786 | 11217 | 311 | 22303 | 96949 |
| 100% 90/10 PGA/PLA (60) | 13052 | UND | 6401 | 49 | 30470 | 2733 | 6499 | 562 | 66825 | 67758 |
| 100% 95/5 PLA/PGA (155) | 11223 | 93 | 3519 | 85 | 67255 | 7085 | 9698 | 1189 | 115744 | 27796 |
| 100% 95/5 PLA/PGA (67) | 2073 | UND | UND | UND | 31242 | 2302 | 4163 | UND | 30645 | 10142 |
| 50% (90/10 PGA/PLA)/50% | 12790 | UND | 12738 | 130 | 107151 | 6172 | 13812 | 253 | 21574 | 146804 |
| PDS (250) | | | | | | | | | | |
| 50% (90/10 PGA/PLA)/50% | 6567 | UND | 3864 | 29 | 27770 | 2564 | 4753 | 517 | 52299 | 37825 |
| PDS (100) | | | | | | | | | | |
| TCP | 2970 | 16 | 552 | 18 | 14848 | 1913 | 3542 | 264 | 201680 | 4268 |

TABLE 3
ELISA results expressed as average fold change compared to tissue
culture plastic. The fold change compared to tissue culture plastic was calculated
from the normalized ELISA results of Table 2. Samples labeled ‘UND’ indicate
that the analyte level was below the detectable limit of the ELISA assay.

| | hTGFB1 | hGCSF | hGMCSF | hIL4 | hVEGF | hHGF | hMCSF | hFGFb | hMMP7 | hIL6 |
|---|---|---|---|---|---|---|---|---|---|---|
| Substrate | fold change | | | | | | | | | |
| 100% 90/10 PGA/PLA | 6.9 | 21.9 | 35.9 | 12.6 | 9.7 | 4.1 | 6.8 | 5.0 | −8.4 | 28.0 |
| (300) | | | | | | | | | | |
| 100% 90/10 PGA/PLA | 6.3 | 5.7 | 19.3 | 2.0 | 4.4 | 2.5 | 3.2 | 1.2 | −9.0 | 22.7 |
| (150) | | | | | | | | | | |
| 100% 90/10 PGA/PLA (60) | 4.4 | UND | 11.6 | 2.6 | 2.1 | 1.4 | 1.8 | 2.1 | −3.0 | 15.9 |
| 100% 95/5 PLA/PGA (155) | 3.8 | 5.8 | 6.4 | 4.6 | 4.5 | 3.7 | 2.7 | 4.5 | −1.7 | 6.5 |
| 100% 95/5 PLA/PGA (67) | 0.7 | UND | UND | UND | 2.1 | 1.2 | 1.2 | UND | −6.6 | 2.4 |
| 50% (90/10 PGA/PLA)/50% | 4.3 | UND | 23.1 | 7.0 | 7.2 | 3.2 | 3.9 | 1.0 | −9.3 | 34.4 |
| PDS (250) | | | | | | | | | | |
| 50% (90/10 PGA/PLA)/50% | 2.2 | UND | 7.0 | 1.6 | 1.9 | 1.3 | 1.3 | 2.0 | −3.9 | 8.9 |
| PDS (100) | | | | | | | | | | |
| TCP | 1.0 | 1.0 | 1.0 | 1.0 | 1.0 | 1.0 | 1.0 | 1.0 | −1.0 | 1.0 |

The results presented in this report support the hypothesis that culturing cells on substrates varying in both composition and density (architecture) alters their cytokine production profile, thus allowing one to use substrates to increase cytokine concentrations in conditioned medium. This is evident in results showing that many of the cytokines examined in this study displayed increased factor production compared to standard tissue culture plastic (TCP). The observed increase in cytokine production is dependent on the composition of the non-woven substrate used, and also to a lesser extent on the density of the non-woven scaffold. When comparing analyte production by cells grown on substrates of similar density (i.e. 100% 90/10 PGA/PLA 150 mg/ml vs. 100% 95/5 PLA/PGA 155 mg/ml) differences observed were dependent on the analyte. In some cases the 100% 90/10 PGA/PLA displayed higher analyte production, in others the 100% 95/5 PLA/PGA displayed higher production, and other analytes displayed no difference between the two. However, a direct comparison of the 50% (90/10 PGA/PLA)/50% PDO nonwovens was more difficult to analyze, as the density of the samples of this composition were not as similar. This will be addressed in the second study. Density also plays a role in these observations, as the samples showing the highest analyte production per cell were the substrate samples of the highest density.

### EXAMPLE 2

### Method of Making Cell Culture Media on Nonwoven Substrates

This example illustrates that culturing cells on non-woven substrates increases the production of trophic factors, altering the composition of the resulting conditioned medium in a positive manner. The observations are a result of both the substrate composition as well as the fabric density.

### Materials & Methods

### Cell Seeding and Culture

This experiment is a repeat of the first example, substituting the CyQuant NF assay kit procedure for the trypsinization procedure used to generate the cell numbers for normalization. Circular samples of one centimeter in diameter were made from non-woven fabric of various compositions. The non-woven substrates chosen are shown in Table 4. Note the highlighted samples were changed from those used in the first study in order to enable a more direct comparison of materials with similar densities. The samples were placed in low-cluster 24-well plates and sterilized by soaking in 100% ethanol for four hours. The samples were then washed with phosphate-buffered saline (PBS—Invitrogen) and placed in renal epithelial growth medium (REGM—Lonza, Walkersville).

TABLE 4
Non-woven substrates assayed. One centimeter circular samples were
created from each of the listed substrates. Highlighted samples were
different than those in the first study and were changed to keep the
density more consistent between substrates of different compositions.

| | Density | Thickness | |
|---|---|---|---|
| Composition | (mg/ml) | (mm) | Lot # |
| 100% 95/5 PLA/PGA | 67 | 1 | 5213-34-1 |
| 100% 95/5 PLA/PGA | 155 | 1 | 5213-34-4 |
| 100% 90/10 PGA/PLA | 60 | 1 | MD00287 rev2 |
| 100% 90/10 PGA/PLA | 150 | 1.5 | MD00092 rev1 |
| 100% 90/10 PGA/PLA | 300 | 1.5 | MD00082 rev3 |
| 50% (90/10 | 150 | 1.5 | MD00015 rev3 |
| PGA/PLA)/50% PDO | | | |
| 50% (90/10 | 323 | 1 | JJ-1-5-2 |
| PGA/PLA)/50% PDO | | | |

Cells (hKDC lot 032906p7) were thawed, counted and brought to 20,000 cells/ml in REGM. The medium was aspirated from the substrates and one ml of the cell suspension (20,000 cells) was added to the wells containing the nonwoven substrate. In addition, 24-well tissue culture plates were seeded as a control (tissue culture plastic). The cell seeded nonwoven scaffolds and control wells were cultured for seven days with fresh medium exchange every two to three days.

### ELISA Analysis of Trophic Factor Secretion

On day seven post-seeding, the cell-seeded materials were washed twice with (PBS) (1 ml/wash), and 500 μl of serum free, phenol-free Dulbecco's Modified Eagles Medium (DMEM) (Invitrogen) supplemented with 2 nM Glutamax (Invitrogen) was added. The cell-seeded substrates were then cultured overnight to generate conditioned medium. The next day, the conditioned medium was collected into 1.5 ml low-retention tubes, centrifuged to remove cells and cell debris, and frozen at −80° C. The concentration of ten specific protein analytes (hM-CSF, hGM-CSF, hIL-4, hIL-6, hVEGF, hIL-13, hHGF, hFGFβ, hMMP-7, hTGFβ1) was determined using the Searchlight analysis service (Pierce Biotechnology, Rockford, Ill.).

After removing the conditioned medium, the cell-seeded substrates were washed with PBS and the number of cells on each nonwoven sample determined using the CyQuant NF assay kit. Briefly, the cultured cells were lysed by the addition of 500 μl CyQuant reagent for one hour at 37° C. In addition, a standard curve ranging from 40,000 to 1,000 cells/well was generated, and lysed by mixing 250 μl of cells in HBSS with 250 μl 2× CyQuant reagent in 24-well plates and incubating alongside the nonwoven samples. Following the lysis incubation, 100 μl from each well was added to a 96-well plate. To be sure the fluorescence was within range of the standard curve, the lysates from the substrates were also diluted 1:5 with 1× CyQuant reagent in the same 96-well plate. Fluorescence was then measured using a SpectraMAX fluorometer (Molecular Devices). The standard curve was used to calculate the number of cells harvested from the substrates. The resulting cell number determinations were subsequently used to normalize the protein analyte concentrations.

### Cell Attachment and Growth

hKDCs were seeded onto one centimeter diameter samples of various non-woven substrates differing in composition and density in triplicate and cultured for seven days. Conditioned medium was then harvested and sent for ELISA analysis. Cells growing on the substrates were recovered using the CyQuant NF assay (Invitrogen). FIG. 2 shows the average cell number determined. The numbers of cells was relatively the same for all substrates tested. In comparison to the cell number results of the first study, the trend observed in which lower density samples supported increased cell numbers was only slightly apparent in the current experiment. Once again, tissue culture plastic supported many more cells than any of the non-woven substrates tested.

### Analysis of Trophic Factor Secretion

Conditioned media samples were analyzed by ELISA and the concentration of 10 analytes was determined. The resulting analyte protein concentrations were normalized to the cell number recovered from the specific samples. Table 5 shows the normalized protein concentration for all analytes and substrates. In general, the expression of most analytes was increased when cells are grown on substrates versus tissue culture plastic (TCP). Only the hMMP-7 analyte was expressed at a higher concentration by cells growing on tissue culture plastic than the substrates. Also, an important trend was observed indicating that the density of the non-woven substrate greatly affects the analyte production per cell. The normalized data show that for most analytes examined, increasing the density of the substrate resulted in an increase in analyte production per cell. Examples of this observation include hVEGF, hHGF, GM-CSF, and M-CSF. When comparing nonwoven substrates of similar density (˜150 mg/ml), the 90/10 PGA/PLA usually displayed the highest analyte production, followed by 50% 90/10 PGA/PLA/PDS, then the 95/5 PLA/PGA samples (see IL-6, M-CSF, GM-CSF). However, this was not always the case—for analytes hTGFβ-1 and hMMP-7, the 100% 95/5 PLA/PGA expression was higher than the 50% (90/10 PGA/PLA)/50% PDO and both samples, respectively.

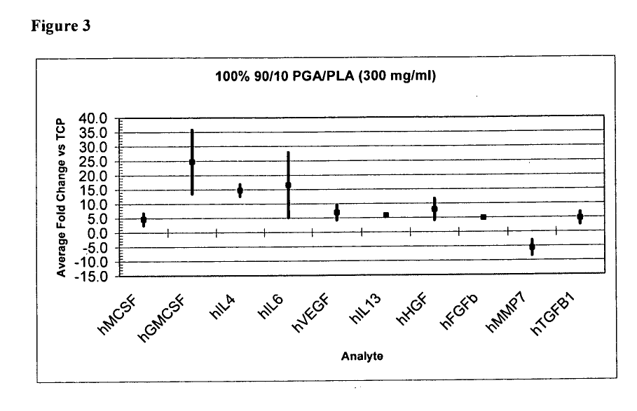

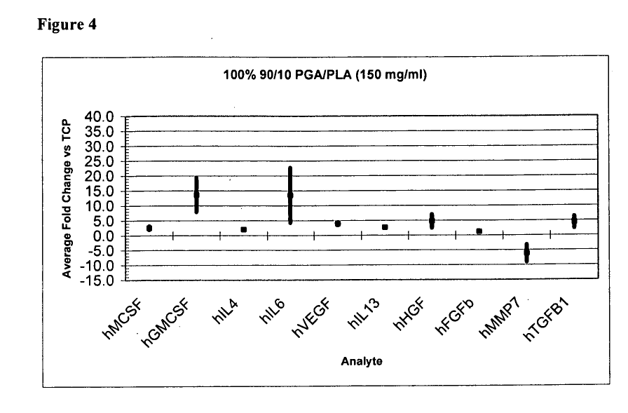

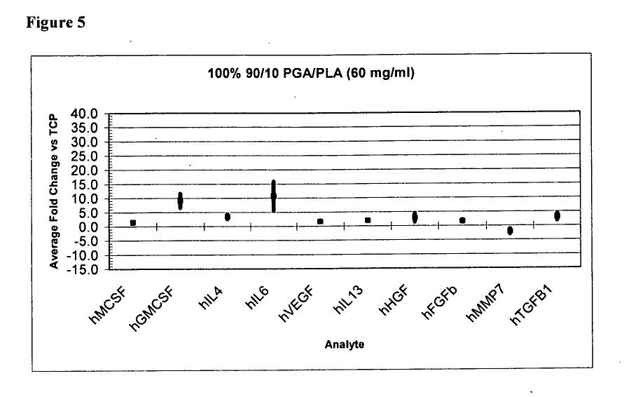

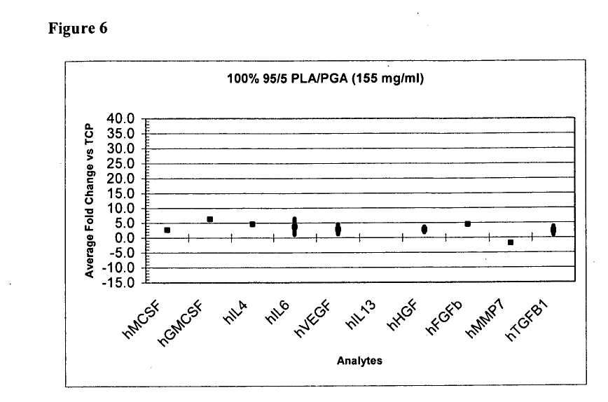

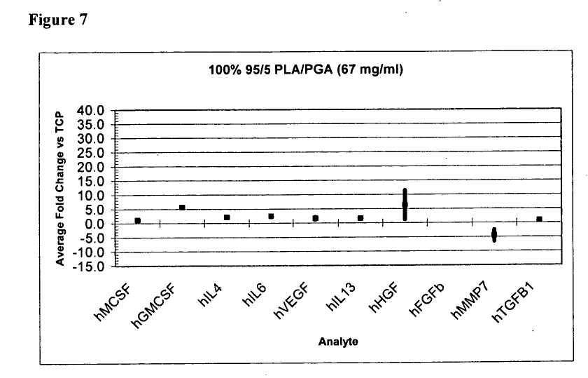

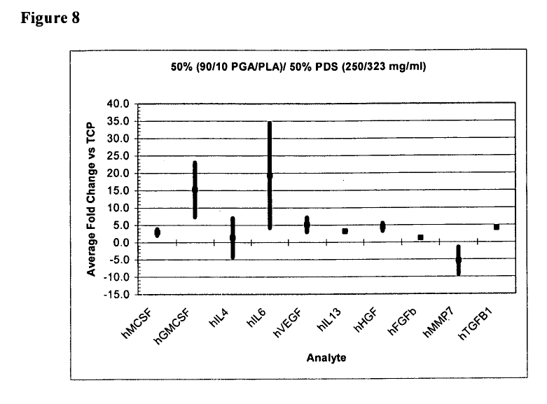

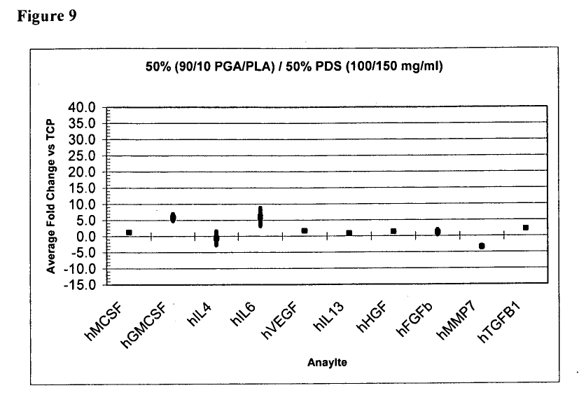

Composition of the non-woven substrates also affected analyte production. These affects are more apparent when the data is expressed as average fold change compared to TCP. Table 6 and FIGS. 3-9 show the observed ranges of analyte expression fold change for each substrate compared to TCP, in table and graphical format, respectively. Looking at FIGS. 3-9, one can easily observe that the 90/10 PGA/PLA non-woven samples produced the greatest fold change, while the 95/5 PLA/PGA samples showed more moderate increases compared to TCP.

TABLE 5
Searchlight analysis of conditioned medium. Conditioned medium
generated from hKDCs cultured on various non-woven substrates were analyzed
using the Searchlight protein array. Data represent the mean analyte
concentrations of duplicate samples normalized to cell number (pg/ml/10⁶ cells).
Data is shown in graphical form in FIG. 2.

| Non-Woven | hMCSF | hGMCSF | hIL4 | hIL6 | hVEGF | hIL13 | hHGF | hFGFb | hMMP7 | hTGFB1 |
|---|---|---|---|---|---|---|---|---|---|---|
| Substrate & (density) | pg/ml/10e6 cells | | | | | | | | | |
| 100% 90/10 PGA/PLA (300) | 31987 | 21612 | 75 | 124572 | 75723 | 2318 | 45918 | <1 | 34005 | 25031 |
| 100% 90/10 PGA/PLA (150) | 24768 | 12736 | <1 | 104788 | 61851 | 1058 | 27401 | <1 | 31702 | 24850 |
| 100% 90/10 PGA/PLA (60) | 14409 | 10674 | 19 | 137098 | 26744 | 791 | 17503 | 1656 | 87658 | 19052 |
| 100% 95/5 PLA/PGA (155) | <1 | <1 | <1 | 22815 | 19900 | <1 | 6731 | <1 | 55414 | 8970 |
| 100% 95/5 PLA/PGA (67) | <1 | 9133 | 10 | 64976 | 22380 | 678 | 45086 | <1 | 40512 | 9923 |
| 50% (90/10 PGA/PLA)/50% | 24625 | 11782 | −19 | 101151 | 54036 | 1258 | 21836 | 2000 | 68157 | 40683 |
| PDS (323) | | | | | | | | | | |
| 50% (90/10 PGA/PLA)/50% | 14620 | 7556 | −12 | 76134 | 28125 | 376 | 5301 | 405 | 34486 | <1 |
| PDS (150) | | | | | | | | | | |
| TCP | 12452 | 1600 | 4 | 24218 | 17986 | 393 | 3923 | 1370 | 102746 | 10384 |

TABLE 6
Combined ELISA results expressed as average fold change compared to
tissue culture plastic. The fold change compared to tissue culture plastic was
calculated from the normalized ELISA results from two studies. Data represent
the fold change from the first experiment, the fold change from the second
experiment, and the average fold change and is written as: Exp1/Exp2/Average.
Data is shown in graphical form in FIG. 3.

| Non-Woven | | | | | | | | | | |
|---|---|---|---|---|---|---|---|---|---|---|
| Substrate & | hMCSF | hGMCSF | hIL4 | hIL6 | hVEGF | hIL13 | hHGF | hFGFb | hMMP7 | hTGFB1 |
| (density) | Fold Change vs TCP: Exp1/Exp2/Average | | | | | | | | | |
| 100% 90/10 | 6.8/2.6/ | 35.9/13.5/ | 12.6/16.9/ | 28/5.1/16.5 | 9.7/4.2/6.9 | NA/5.9/5.9 | 4.1/11.7/7.9 | 5/NA/5 | −8.4/−3/−5.7 | 6.9/2.4/4.7 |
| PGA/PLA | 4.7 | 24.7 | 14.7 | | | | | | | |
| (300) | | | | | | | | | | |
| 100% 90/10 | 3.2/2/ | 19.3/8/13.6 | 2/NA/2 | 22.7/4.3/13.5 | 4.4/3.4/3.9 | NA/2.7/2.7 | 2.5/7/4.7 | 1.2/NA/1.2 | −9/−3.2/−6.1 | 6.3/2.4/4.3 |
| PGA/PLA | 2.6 | | | | | | | | | |
| (150) | | | | | | | | | | |
| 100% 90/10 | 1.8/1.2/ | 11.6/6.7/9.1 | 2.6/4.3/3.5 | 15.9/5.7/10.8 | 2.1/1.5/1.8 | NA/2/2 | 1.4/4.5/2.9 | 2.1/1.2/1.7 | −3/−1.2/−2.1 | 4.4/1.8/3.1 |
| PGA/PLA | 1.5 | | | | | | | | | |
| (60) | | | | | | | | | | |
| 100% 95/5 | 2.7/na/ | 6.4/NA/6.4 | 4.6/NA/4.6 | 6.5/0.9/3.7 | 4.5/1.1/2.8 | NA | 3.7/1.7/2.7 | 4.5/NA/4.5 | −1.7/−1.9/−1.8 | 3.8/0.9/2.3 |
| PLA/PGA | 2.7 | | | | | | | | | |
| (155) | | | | | | | | | | |
| 100% 95/5 | 1.2/NA/ | NA/5.7/5.7 | Na/2.2/2.2 | 2.4/2.7/2.5 | 2.1/1.2/1.7 | NA/1.7/1.7 | 1.2/11.5/6.3 | NA | −6.6/−2.5/−4.6 | 0.7/1.0/0.8 |
| PLA/PGA | 1.2 | | | | | | | | | |
| (67) | | | | | | | | | | |
| 50% (90/10 | 3.9/2.0/ | 23.1/7.4/15.2 | 7.0/−4.3/1.4 | 34.4/4.2/19.3 | 7.2/3.0/5.1 | NA/3.2/3.2 | 3.2/5.6/4.4 | 1.0/1.5/1.2 | −9.3/−1.5/−5.4 | 4.3/3.9/4.1 |
| PGA/PLA)/ | 2.9 | | | | | | | | | |
| 50% PDS | | | | | | | | | | |
| (323) | | | | | | | | | | |
| 50% (90/10 | 1.3/1.2/ | 7.0/4.7/5.9 | 1.6/−2.8/ | 8.9/3.1/6.0 | 1.9/1.6/1.7 | NA/1.0/1.0 | 1.3/1.4/1.3 | 2.0/0.3/1.1 | −3.9/−3.0/−3.4 | 2.2/NA/ |
| PGA/PLA)/ | 1.3 | | −0.6 | | | | | | | 2.2 |
| 50% PDS | | | | | | | | | | |
| (150) | | | | | | | | | | |
| Tissue | 1 | 1 | 1 | 1 | 1 | 1 | 1 | 1 | 1 | 1 |
| Culture | | | | | | | | | | |
| Plastic | | | | | | | | | | |

The results presented in this report support the hypothesis that culturing cells on non-woven substrates varying in both composition and density (architecture) alters their cytokine production profile, thus allowing one to use biomaterials to increase cytokine concentrations in produced conditioned medium. This is evident in results showing that many of the cytokines examined in this study displayed increased factor production compared to standard tissue culture plastic (TCP). The observed increase in cytokine production is dependent on the composition of the non-woven substrate used, and also to a lesser extent on the density of the non-woven scaffold. The effect of substrate composition is most apparent in FIGS. 3-9, in which the observed fold changes for the analytes are graphed together for each composition tested. The 90/10 PGA/PLA non-woven samples display the largest increases in analyte production compared to TCP, followed by the 90/10 PGA/PLA/PDS blend, with the 95/5 PLA/PGA samples producing the lowest increase, which in most cases was 2-fold greater than TCP. When comparing analyte secretion by cells grown on substrates of similar density (i.e. ˜150 mg/ml), differences observed were analyte specific.

This example differs in the procedure used to determine the number of cells attached to the substrates for normalization from Example 1. However, the use of an alternate method of determining cell number in this report compared to the first study resulted in comparable results. This eliminates concern regarding the efficiency of cell recovery from samples of different density (and cell penetration) and it's effects on the normalization of the data.

We claim:

1. A method of making conditioned media comprising the steps of:
a. providing a population of mammalian kidney-derived cells,
b. seeding the cells on a nonwoven substrate,
c. culturing the cells on the nonwoven substrate in renal growth medium,
d. removing the renal growth medium,
e. culturing the cells on the nonwoven substrate in serum free medium for about 24 hours, and
f. isolating the conditioned medium from the cell culture.

2. The method of claim 1, wherein the density of the nonwoven substrate is from about 60 mg/mL to about 350 mg/mL.

3. The method of claim 2, wherein the nonwoven substrate is comprised of an aliphatic polyester fibers.

4. The method of claim 3, wherein the aliphatic polyester fiber is comprised of homopolymers or copolymers of lactide (which includes lactic acid D-,L- and meso lactide), glycolide (including glycolic acid), epsilon-caprolactone, p-dioxanone (1,4-dioxan-2-one), trimethylene carbonate (1,3-dioxan-2-one), and combinations thereof.

5. A conditioned media prepared by the method comprising the steps of
a. providing a population of mammalian kidney-derived cells,
b. seeding the cells on a nonwoven substrate,
c. culturing the cells on the nonwoven substrate in renal growth medium,
d. removing the renal growth medium,
e. culturing the cells on the nonwoven substrate in serum free medium for about 24 hours, and
f. isolating the conditioned medium from the cell culture.

6. The conditioned media prepared by the method of claim 5 wherein the density of the nonwoven substrate is from about 60 mg/mL to about 350 mg/mL.

7. The conditioned media prepared by the method of claim 6, wherein the nonwoven substrate is comprised of an aliphatic polyester fibers.

8. The conditioned media prepared by the method of claim 7, wherein the aliphatic polyester fiber is comprised of homopolymers or copolymers of lactide (which includes lactic acid D-,L- and meso lactide), glycolide (including glycolic acid), epsilon-caprolactone, p-dioxanone (1,4-dioxan-2-one), trimethylene carbonate (1,3-dioxan-2-one), and combinations thereof.
